(19) United States
(12) Patent Application Publication
(10) Pub. No.: US 2020/0023022 A1
(43) Pub. Date: Jan. 23, 2020

(54) NUTRITIONAL SUPPLEMENT COMPRISING MICROALGAE, VITAMINS AND MINERALS

(71) Applicant: Stefano Scoglio, (US)

(72) Inventor: Stefano Scoglio, (US)

(21) Appl. No.: 15/781,145

(22) PCT Filed: Dec. 4, 2016
(86) PCT No.: PCT/EP2016/079690

§ 371 Date: Jun. 3, 2018

(51) Int. Cl. A61K 35/748; A61K 36/752; A61K 36/22; A61K 36/28; A61K 36/185; A61K 36/75; A61K 36/05; A61K 36/31; A61K 36/61; A61K 36/06; A61P 3/02; A23L 17/60; A23L 33/105; A23L 33/15; A23L 33/155; A23L 33/16

(52) U.S. Cl. CPC A61K 35/748; A23V 2002/00; A61K 36/22; A61K 36/28; A61K 36/185; A61K 36/75; A61K 36/05; A61K 36/31; A61K 36/61; A61K 36/06; A61P 3/02; A23L 17/60; A23L 33/105; A23L 33/15; A23L 33/155; A23L 33/16; A61K 36/752

(57) ABSTRACT

Multivitamins and multi-mineral supplements are widely used as a remedy for lack of proper nutrition. However, they tend to be scarcely assimilated, and more and more scientific reports claim that they are relatively useless if not harmful. This invention is an answer to this problem, as it provides a novel product, based on the nutritional bounty of cyanobacterial blue-green algae, and reinforced by foods and herbal extracts rich in one or more of the vitamins or minerals missing or scarce in the cyanobacterial blue-green algae. The result is a multivitamin and multi-mineral supplement that is completely organic and, being from alimentary and herbal sources that are recognized as foods by our organism, are much better assimilated. This invention provides an optimal solution to the conundrum we face today: the standard contemporary diet does not provide enough nutrition, with negative consequences on our health; but the inorganic and synthetic supplements we consume are not really foods, and so are ineffective. The product we have invented is an effective and innovative answer to this problem.

### BACKGROUND OF THE INVENTION

The market of nutritional supplements, that is of multivitamin and multimineral supplements, is worth billions of dollars worldwide, based on the widespread idea that modern nutrition, even in advanced industrial and rich societies, is lacking due to different factors: a) foods today have generally a low nutrient content due to the long-standing industrial and monocultural way of farming, which has impoverished agricultural soil and consequently the food that in that soil are produced; b) the distribution chain of fresh fruit and vegetables, which involves long distance traveling, days of refrigeration and other conservation techniques, further increases the loss of nutrients in agricultural foods; c) our own contemporary habits, whereby most people rarely have a proper meal sitting at a table, makes it even more difficult for many people to ingest sufficient quantities of the required nutrients.

This historical situation has generated epidemiological researches around the world, from which it can be inferred that only a percentage of people has actually a complete nutrition (Pennington et al., 1991, Syrad et al., 2016), with all the vitamins and minerals required for good health; and many think that this is the real cause behind the contemporary increase in metabolic and degenerative disorders and diseases.

This conviction seems to have taken hold of people's mind, and it is the main cause behind the widespread use of multivitamins and multi-minerals. However, in the last 15 years, more and more reports have claimed that such supplements are at best relatively useless, and at worst even harmful (Omenn, 1996; Heart Protection Study, 2002; Miller et al. 2005). A main reason for such claims, which are generally backed by solid scientific research, is that such multivitamins and multi-minerals are of inorganic and synthetic origin, thus are not properly absorbed by our bodies, and can even accumulate to the point of creating damages in the organism.

The alternative has been that of using different types of so called superfoods, that is foods that are natural, organic and thus digestible and absorbable by the human organism, yet naturally endowed with an uncommonly higher nutritional content. Some herbs come to mind, such as Siberian Ginseng or Maca. But these are mainly advocated as adaptogens, with some relevant but not so rich nutritional content.

Another type of nutritionally dense superfoods are the green grasses from grains such as wheat, barley, alfa alfa, and so on. The specific quality of such grasses is that at the grass level nutrients are still intact and available, and have not been used by the plant to build up the seeds and grains. Moreover, at the grass level, the plant has still not produced anti-nutritional factors such as phytic acid, which prevents the absorption of minerals, or proteins very difficult to digest such as gluten. Overall, then, cereal grasses are a relevant superfood.

However, in terms of nutrient concentration, the most nutrient rich superfoods are by far the microalgae, and especially, but not only, the cyanobacteria. The most widely known are the cyanobacteria Spirulina and Aphanizomenon flos aquae (Klamath AFA), and the unicellular non-cyanobacterial Chlorella. When compared with the cereal grasses, their nutritional content is far superior. Even in terms of just chlorophyll content, while the cereal grasses have an approximate 0.3% chlorophyll content, that of the microalgae ranges from 1% to 1.5%, that is up to 5 times more.

When looking at the actual nutritional content of the 3 microalgae, however, we find that they are rich in certain nutrients, and not rich enough in others. But what are the criteria to establish what is nutritionally rich, or even a source of that certain nutrient? There are different approaches to this, usually following the different national definitions from the different national authorities. For instance, the following is the table of nutrients daily requirements (Nutrient Reference Values) established by the European Community (UE 1169/2011, XIII, A, 1) on the one hand, and by the United States National Academy of Sciences on the other:

| | EUROPEAN | | |
|---|---|---|---|
| | NRVs 2011 | USA DRIs | |
| VITAMINs | | | |
| vitamin A | mcg 800 | 900 | mcg |
| vitamin D | mcg 5 | 5 | mcg |
| vitamin E | mg 12 | 15 | mg |
| vitamin K | mcg 75 | 120 | mcg |
| vitamin C | mg 80 | 90 | mg |
| thiamin (vitamin B1) | mg 1.1 | 1.2 | mg. |
| riboflavin (vitamin B2) | mg 1.4 | 1.3 | mg |
| niacin | mg 16 | 16 | mg |
| vitamin B6 | mg 1.4 | 1.3 | mg |
| folacin (folic acid) | mcg 200 | 400 | mcg |
| vitamin B12 | mcg 2.5 | 2.4 | mcg |
| biotin | mcc 50 | 0.030 | mg |
| panthotenic acid | mg 6 | 5 | mg. |
| MINERALS | | | |
| calcium | mg 800 | 1000 | mg |
| phosphorous | mg 700 | 700 | mg |
| iron | mg 14 | 8 | mg |
| magnesium | mg 375 | 400 | mg |
| zinc | mg 10 | 11 | mg |
| iodine | mcg 150 | 150 | mcg. |
| copper | mg 1 | 0.9 | mg |
| fluoride | mg 3.5 | 4 | mg |
| selenium | mcg 55 | 55 | mcg |
| manganese | mg 2 | 2.3 | mg |
| chromium | mcg 40 | 35 | mcg |
| molibdenum | mcg 50 | 45 | mcg |

| | | |
|---|---|---|
| boron | mg 1 | value not established |
| Chlorine, sodium, potassium | 800/—/2000 | 2300/1500/4700 mg |
| other trace minerals (nichel, | value not | value not established |
| stagno, silica) | established | |

As shown by the table, only a certain number of nutrients are provided with a daily requirement of NRV/DRI. All the vitamins have a daily requirement; whereas only a handful of minerals have one. Excluded are mainly the trace minerals, which are nutritionally very important, to the point that in the past the US Department of Agriculture performed studies showing how essential they were, but managed only to have them recognized as “quasi-essential”, which is another way of saying, yes, they are very important, but for most of them we cannot really establish an official DRI (except a few, such as chromium both in the EU and in the US). In any case, even without an established NRV/DRI, and considering that as trace minerals they may be effective at infinitesimal quantities, when talking of superfoods, the quality of having a wide trace mineral spectrum can be defined as “quasi-essential”.

When comparing the EU NRVs and the US DRIs, we see that the picture is slightly different, but not so much. Some nutrients's requirements are higher in the EU, some others are higher in the US. Probably, an average between the two lists is the proper, valid reference.

Now, it is a known fact that the inorganic minerals and synthetic vitamins used in standard nutritional supplements are poorly absorbed and so relatively useless, yet this truth is still not fully admitted by health authorities, and so one can find synthetic supplements that can satisfy the daily requirements in little quantities of the product, but at the cost of absorption and therefore efficacy. For example, if you use pure ascorbic acid as vitamin C, you can put 80 mg. of it in a tablet, and satisfy the EU NRV 100%; while if you used a natural vitamin C source, such as an Acerola's extract containing 34% of vitamin C, you would need to use 235 mg. of the extract, that is almost three times as much as with synthetic ascorbic acid. But there is no comparison about the absorption and efficacy of the two.

Therefore, any natural nutritional supplement, that is any nutritional supplement composed of organic nutrients from natural sources, will have the disadvantage of requiring overall a higher dosage. However, it will have the much more important advantage that the nutrients it will provide will be absorbed by our body, thus achieving a truly beneficial health effect.

The basic invention presented here solves the problem of how to formulate a nutritional supplement that, while being formed exclusively by nutrients derived by natural and organic sources, will provide quantities of such nutrients that can be defined as relevant for the daily requirements. The notion of daily requirement relevance in relation to each specific nutrient can be defined as the ability of a certain product to provide the consumer, in the chosen dosage, at least 15% (according to EU and Italian standards), and possibly up to 100% and more of the established NRV/DRI. We will call such relevance of the amount of natural nutrient contained in the product “RDA-relevance”, using the old acronym used for the daily requirement (RDA=Recommended Daily Allowance).

The product that we have invented provide RDA-relevant and natural nutrients in reference to almost all of the nutrients for which there is an established NRV/DRI, while offering the further advantage of providing, thanks to the basic microalgal content, the whole array of the quasi-essential trace minerals for which no value has yet been established.

In order to achieve that goal, our invention provides the combination of: a) a microalga such as Aphanizomenon flos aquae (AFA) and/or Spirulina, as the best possible source of a wide spectrum of organic vitamins and minerals; b) natural sources of each specific organic nutrient whose presence in the microalgae is insufficient to provide RDA-relevant quantities.

Before moving to the disclosure of the actual invention, it is important to explain why, even though our invention covers all microalgae as a possible base for an organic and food base multivitamin and multivitamins supplement, the invention takes Aphanizomenon flos aquae as its preferred embodiment. The following is a table comparing the nutritional content of the 3 main microalgae used for human consumption, namely AFA from Klamath Lake, Spirulina and Chlorella:

Comparative table of AFA¹, Spirulina² and Chlorella³

| | | | |
|---|---|---|---|
| Vitamins | AFA | Spirulina | Chlorella |

Vitamins content in 1 g.

| Vitamin A (as beta-carotene) | 600-800 | μg | 600-800 | μg | 21 | μg |
|---|---|---|---|---|---|---|
| Vitamin B1 (thiamine) | 200-250 | μg | 25-30 | μg | 10-18 | μg |
| Vitamin B2 (riboflavine) | 35-40 | μg | 35-40 | μg | 40-45 | μg |
| Vitamin B3 (niacine) | 4000 | μg | 150 | μg | 180-200 | μg |
| Vitamin B4 (choline) | 1700 | μg | Unreported | | Unreported | |
| Vitamin B5 (pantothenic acid) | 1500 | μg | 1 | μg | 90 | μg |
| Vitamin B6 (pyridoxine) | 25-30 | μg | 8 | μg | 18-20 | μg |
| Vitamin B9 (folic acid) | 40 | μg | 0.1 | μg | 25 | μg |
| Vitamin B12 (cobalamine) | 4-6 | μg | 1 | μg | 1 | μg |
| Vitamin C | 2000 | μg | 0 | | 70 | μg |
| Vitamin E | 280 | μg | 16 | μg | 50 | μg |

| | | | | | |
|---|---|---|---|---|---|
| Vitamin K | 70 | μg | 20 | μg | traces |

¹Klamath CD ®Max
²EarthFood Spirulina
³SunChlorella

The table shows that, apart from the content of beta-carotene which is substantially similar between Spirulina and AFA, for every other vitamin, from the vitamin B-complex to vitamin K, AFA has nutritional values that are far superior relative to the other 2 microalgae. On the other hand, AFA alga is relatively scarce, and it may be necessary, in case of success of the product, to make use also of one or both of Spirulina and Chlorella as substitutes or in association with AFA. Here we report a nutritional multi-vitamin and multi-mineral supplement based on AFA algae plus other organic food sources of RDA-relevant nutrients.

### DISCLOSURE OF THE INVENTION

The natural organic multi-vitamins and multi-minerals supplements will be manufactured in two or more possible formats:
a) a bottled supplement of 30, 60, 90 or more capsules or tablets, with the capsules being of the 0 or 00 type; and the tablets being of 500 mg. to 1 gram;
b) a supplement formed by sachets, each having the whole or half of the daily dosage of the supplement;
c) a liquid drink containing dissolved powder providing enough of the combination of food source nutrients to be RDA-relevant.

Let's thus look at each nutrient, to see how the combination of microalgae, especially AFA, with one or the other high concentrated source of the same nutrient can help our novel nutritional supplement to reach an RDA-relevant status for most nutrients.

The reason why AFA can constitute the fundamental base of an organic, food grade nutritional supplement is due to the fact that altogether it is the food that has the widest spectrum of RDA-relevant nutrients, so that it becomes possible to add few other food or herbal extracts to the basic 2 grams of algae (more or less) to obtain a product capable of providing a wide spectrum of RDA-relevant nutrients in their natural and thus assimilated food form.

Vitamins

Vitamin A

AFA algae at its best, contains approximately 200 mcg. of vitamin A×gram, so 2 grams of AFA a day would provide about 400 mcg. or approximately 50% of the RDA/LARN. In reality, vitamin A as such is contained only in animal and dairy foods, while vegetable sources contain carotenes that act as vitamin A precursors. AFA algae and Spirulina are such sources of carotenes, as is the microalgae Dunaliella salina. There are other good food and herbal sources of food grade vitamin A that could be used, such as Annatto (Bixa orellana) seeds, but few pack such high amounts of vitamine A precursors such as the microalgae. So, our best choice would be an extract from the microalga Dunaliella salina, with a 7.5% betacarotene concentration. The numbers given below are thus obtained: 100 mg. of Dunaliella extract contains 7.5 mg. of betacarotene; to convert betacarotene into vitamin A we divide such quantity by 6, thus obtaining 1.250 mg. of vitamin A. The same rate of conversion is applied to the AFA algae's carotenes.

| | Dunaliella extract | AFA algae | | |
|---|---|---|---|---|
| Vitamin | 100 mg. | 2 grams | Total | % RDA |
| Vitamin A as | 750 mcg. | 400 mcg. | 1,150 mg. | 127% |
| betacarotene | | | | |

B Vitamins

Again, AFA algae provides in 2 grams substantial quantities of the main B vitamins, and more specifically (RDAs are calculated on the EU standards):

| | | |
|---|---|---|
| B1 - | 500 mcg. | 45.5% RDA |
| B2 - | 75 mcg. | 5.3% RDA |
| B3 - | 7 mg. | 38.8% RDA |
| B5 - | 3 mg. | 43% RDA |
| B6 - | 54 mcg. | 3.8% RDA |
| B9 - Folic acid | 80 mcg. | 40% RDA |
| B12 - Cobalamine | 2.5 mcg.¹ | 100% RDA |

¹The actual content of B12 in AFA is between 6 to 8 mcg. x gram. However, a substantial percentage of AFA's B12 is formed by cobalamine analogues, only about 20% of it being true cobalamine, we calculated that the actual B12 is about 2.5 mcg. in 2 grams of algae. For its absorption, see: Baroni L. et al, 2009.

We see that AFA algae is probably the best source of organic, food grade B vitamins overall. However, apart from B12 at 100%, the other B vitamins are some RDA-relevant (that is they provide a minimum of 15% of the RDA), some others, such as B2 and B6, are non RDA-relevant. How to supplement those specific vitamins from other food sources? Particularly good sources of B-vitamins are avocado, pomegranate, dates, watermelon, and some berries; leafy greens and vegetables such as amaranth, Swiss chard, kale, Brussels sprouts, potatoes, squashes and parsnips also contain significant amounts; among legumes soy beans, black-eye peas and edamame contain high amounts of B9, also known as folate. A possible alternative would be to extract the B-vitamin complex from the very cyanobacterial micro algae such as Klamath algae and/or Spirulina, or from fruits such as Lemon, Guava and Mango, or herbs such as Holy Basil. Thus, a blend of extracts from these foods can produce a high concentrated spectrum of B-vitamins. Just as an example, we have performed a water extraction from a blend of different foods, namely Klamath microalga, Lemon, and Mango, and by adding just 180 mg. of such an extract blend to the basic 2 grams of AFA in the daily dosage we obtain the following values:

| | AFA & Fruits | | | | | | |
|---|---|---|---|---|---|---|---|
| B Vitamins | extract | | Whole AFA | | Daily dose | | % NRV |
| B1 -Thiamine | 3.6 | mg | 0.5 | mg | 4.1 | mg | 372% |
| B2 - Riboflavin | 3.78 | mg | 0.075 | mg | 3.855 | mg | 275% |
| B3 - Niacin | 6.12 | mg | 7 | mg | 13.12 | mg | 82% |
| B5 - Pantothenic | 6.84 | mg | 3 | mg. | 9.84 | mg. | 164% |
| Acid | | | | | | | |
| B6 - Pyridoxine | 4.32 | mg. | 0.054 | mg. | 4.374 | mg | 312% |
| B9 - Folic Acid | 126 | mcg. | 80 | mcg. | 206 | mcg. | 103% |
| B12 - Cobalamine | 1 | mcg. | 2.5 | mcg. | 3.5 | mcg. | 140% |

As evident from the above table, the daily dose of the AFA based organic nutritional supplement provides all the B-Vitamins in an RDA-relevant quantity. Indeed, with the sole exception of Niacin, at 73% of the RDA, all the other vitamins are given in quantities well above 100% of the minimum daily requirement, leaving thus open the possibility if using lower quantities of the extract, thus also reducing the cost of the product.

Vitamin C

AFA algae is a source of vitamin C, as opposed to other microalgae such as Spirulina. However, its content is limited, and in 2 grams it only provides about 4 mg., vs. an NRV of 80 mg. The sources of natural, organic, food grade vitamin C are many, starting with the Rosa Canina fruit, or the Acerola fruit, or the indian fruit Amla, as well as lemon or other citrus fruit extracts. Any one of such food extracts can be a good integration for the little vitamin C provided by AFA. In the following table an example with the Acerola extract at 34% vitamin C:

| | Acerola extract 34%, | AFA algae | | |
|---|---|---|---|---|
| Vitamin | 225 mg. | 2 grams | Total | % NRV |
| Vitamin C | 76.5 mg. | 4 mg. | 80.5 mg. | 100% |

Vitamin E

Vitamin E is the other vitamin of which AFA is a source, but a poor one in relative terms. In absolute terms, AFA has actually a very high content of vitamin E: in 100 grams, it contains 25 mg., or 250% of the daily requirement. But in just 2 grams, it provides 0.5 mg., or only 5% of the daily requirement. There are a few powder sources of natural vitamin E, and they are generally dried oil based sources (vitamin E being a fatty vitamin). Among those, extracts from soy seeds, sunflower seeds, or Annatto seeds, are the most common. A sunflower seed extract, for instance, provides 15 mg. of vitamin E in just 100 mg.:

| | Sunflower seed | AFA algae | | |
|---|---|---|---|---|
| Vitamin | extract 15% - 80 mg. | 2 grams | Total | % NRV |
| Vitamin E | 12 mg. | 0.5 mg. | 12.5 mg. | 104% |

Vitamin D

Vitamin D is found very scarcely in foods, and can be synthesized by our body from sunlight. However, as more and more people are deprived of natural sunlight, providing a food source of vitamin D can be important. AFA algae may have traces of vitamin D, but they are not really relevant. The concentrated food sources of vitamin D are very few. One of these are mushrooms: a mushroom extract can provide up to 5,000 IU of vitamin D2×gram (equivalent to 125 mcg.). Thus, 50 mg. of such extract can provide approximately 6,25 mcg., which is equivalent to 250% of the Italian RDA, and to 83% of the American RDA:

| | Mushroom extr 5000 | AFA algae | | |
|---|---|---|---|---|
| Vitamin | IU/gr - 50 mg. | 2 grams | Total | % NRV |
| Vitamin D | 6.25 mcg. | 0 mcg. | 6.25 mcg. | 125% |

Vitamin K

Vitamin K is an essential vitamin which, in its natural form, plays a variety of physiological functions, such as blood coagulation and bone formation. Leafy green vegetable are a food source of vitamin K, but so is AFA algae, which provides a whooping 60 mcg./gr., and so in 2 grams 160% of the EU Nutritional Reference Value (NRV) and 100% of the US DRI:

| | | AFA algae | | |
|---|---|---|---|---|
| Vitamin | Other sources | 2 grams | Total | % NRV |
| Vitamin K | 0 mcg. | 120 mcg. | 120 mcg. | 171% |

Minerals

Concerning the ability of the product to provide RDA-relevant quantities of minerals, we need to clarify 3 different aspects:

Macrominerals

Macrominerals such as calcium, magnesium, phosphorous, etc. have such high RDAs quantities that it is impossible to provide RDA-relevant quantities in a few grams of a nutritional supplement. This is why macrominerals need to be supplemented thorough a specific product, and cannot but be excluded from the RDA relevant spectrum of nutrients included here.

Trace Minerals

There is then the list of all trace minerals, for which no daily requirement has been established but that are often considered quasi-essential (Uthus, 1990; Mayer et al., 1993). We don't really need to do anything about these, as AFA algae is actually the best living source of such trace minerals, having their complete spectrum and thus providing trace quantities of petty much all of them, as visible from the table below (ppm—parts×millions):

| | | | | | | | |
|---|---|---|---|---|---|---|---|
| Boron | 11.4 | Calcium | 8100 | Barium | 5.67 | Niobium | 0.08 |
| Aluminum | 38.2 | Chromium | 0.92 | Beryllium | <.01 | Osmium | <.05 |
| Cobalt | 1.28 | Copper | 7.29 | Bismuth | 0.29 | Palldium | <.01 |
| Antimony | 2.48 | Germanium | 0.43 | Bromine | 47.2 | Platinum | <.01 |
| Iodine | 18.3 | Iron | 4190 | Cadmium | 0.107 | Prascodymium | 0.15 |
| Magnesium | 31.9 | Manganese | 24.7 | Carbon | 936,000 | Rhenium | <.05 |
| Molybdenum | 3.77 | Nickel | 6.37 | Cerium | 1.21 | Rhodium | <.01 |
| Phosphorus | 16.2 | Potassium | 15,200 | Cesium | 0.09 | Rubidium | 0.9 |
| Selenium | 0.488 | Silicon | 233 | Chloride | 16,200 | Ruthenium | 0.06 |
| Sodium | 19.2 | Titanium | 0.48 | Mercury | 0.007 | Samarium | 0.85 |
| Tin | 0.12 | Zinc | 2.98 | Neodymium | 0.31 | Scandium | 0.037 |
| Dysprosium | 0.347 | Hafnium | 0.076 | Lutctium | 0.089 | Thallium | 2.85 |
| Erbium | 0.882 | Holmium | 0.054 | Silver | 0.018 | Thorium | <.05 |
| Europium | <.05 | Indium | 0.077 | Strontium | 5.88 | Thulium | 0.095 |
| Fluoride | 872 | Iridium | <.05 | Sulfur | 350 | Tungsten | 0.091 |
| Gadolinium | 0.96 | Lanthanum | <.05 | Tantalum | 0.65 | Vanadium | 1.8 |
| Gallium | 0.82 | Lead | 0.082 | Tellurium | 0.11 | Ytterbium | 0.084 |
| Gold | 0.037 | Lithium | 0.74 | Terbium | 0.071 | Yttrium | 0.192 |
| | | | | | | Zirconium | 0.74 |

Microminerals with a Daily Requirement

There are about 11 microminerals for which a NRV/DRI has been established in the order of milligrams or micrograms. Our novel product manages to provide as many as 9 of these minerals in a natural and organic form and at levels equal or above 100% of the daily requirement.

Chromium

Chromium, like vanadium, plays an important role in the regulation of sugar metabolism, a role that is very important in today's world, with an increasing growth of what is defined as “metabolic syndrome”, and that includes pathologies such as diabetes and obesity. AFA algae contains a little amount of chromium, approximately 2 mcg. in 2 grams; and in order to reach the RDA of 35 mcg. we need to introduce some other foods, such as extracts from grape juice, chromium-rich yeast, and most of all broccoli and other Brassicaceae such as the Moringa plant (and extract of which can provide up to 33 mcg. in just 33 mg. of extract).

| | Moringa Oleifera | AFA algae | | |
|---|---|---|---|---|
| Mineral | extract - 38 mg. | 2 grams | Total | % NRV |
| Chromium | 38 mcg. | 2 mcg. | 40 mcg. | 100% |

Iron

As visible in the table above, AFA algae contains approx. 4 mg/gr. of iron, a very substantial quantity. That in itself represents 50% of the US DRI; and approximately 30% of the EU's NRV. Thus, 2 grams of AFA algae in the formula can provide up to 100% of the US DRI and almost 60% of the EU's NRV. As this quantity may not always be guaranteed, since there may be variations in the iron content of the algae; and as we may need to increase the quantity to reach 100% of the European daily requirement, we can use other sources of organic iron, such as extracts of clams, of certain types of beans, backstrap molasses, or curry plant leaves (Muraya koeinigii). For instance, by adding 200 mg. of a curry plant leaves's extract, we can obtain the following:

| | Muraya koeinigii | AFA algae | | |
|---|---|---|---|---|
| Mineral | leaves extr. 200 mg. | 2 grams | Total | % RDA |
| Iron | 7.2 mg. | 8 mg. | 15.2 mcg. | 108% |

Fluoride

As opposed to the industrially produced sodium fluoride, the calcium fluoride found in nature and in natural foods is essential for bone and teeth formation and maintenance. AFA algae has very high content of natural fluoride, about 1 mg/gr., approximately 57% of the new and heightened NRV, a quantity that is in itself significantly relevant to the satisfaction of the daily requirement.

| | | AFA algae | | |
|---|---|---|---|---|
| Mineral | Other sources | 2 grams | Total | % RDA |
| Fluoride | 0 mg. | 2 mg. | 1.74 mcg. | 57% |

Iodine

Iodine is an essential mineral for metabolism and thyroid function. However, excess iodine may be problematic for people with hyperactive thyroid, and while it is important for a multi-nutrients supplement to provide some iodine, it may be better not to provide too much. AFA algae has a useful content of iodine, which taken daily may be enough in itself, even though it can be eventually supplemented with a specific kelp extract.

| | | AFA algae | | |
|---|---|---|---|---|
| Mineral | Kelp extract 30 mg. | 2 grams | Total | % RDA |
| Iodine | 25 mcg. | 36 mcg. | 61 mcg. | 40% |

Molybdenum

Molybdenum is a trace mineral essential for the activation of many enzymes and for a proper protein metabolism, being crucial to the synthesis of amino acids. Its presence in the diet depends on its presence in the soil, and so its content in specific foods is variable. Good sources are beans and lentils, as well as nuts and dairy, 2 grams of AFA algae provide approximately 16% of the RDA, a quantity sufficient to qualify the product as a good source of molybdenum.

| | | AFA algae | | |
|---|---|---|---|---|
| Mineral | Other sources | 2 grams | Total | % RDA |
| Molybdenum | 0 mg. | 8 mcg. | 8 mcg. | 16% |

Selenium

Selenium is one of the few trace minerals for which there is an official daily requirement. This is due to the fact that many studies have been performed showing the very relevant anti-oxidant properties of this mineral, especially again in its natural and organic form. AFA algae is a source of selenium, but in small quantity, only about 0.5 mcg./gr. We thus need to supplement this quantity with an alternative good natural source. Among the various good vegetable sources, the best ones are Brazilian nuts and Mustard seeds (Brassica nigra). An extract of either one can provide about 0.5%/dw of selenium, thus providing 100% of the RDA in just 11 mg.

| | Brassica nigra seeds - | AFA algae | | |
|---|---|---|---|---|
| Mineral | 12 mg. | 2 grams | Total | % NRV |
| Selenium | 60 mcg. | 2 mcg. | 62 mcg. | 112% |

Vanadium

Vanadium is an essential mineral in relation to the sugar metabolism. It acts in fact as an insulin-mimetic mineral, able to stimulate the insulin receptor, thus reducing the quantity of insulin needed. AFA algae is a good source of vanadium, and thus may not need to be supplemented by any other source; even though it could be enriched by adding extracts from buckwheat or parsley. However, while there was a daily requirement for vanadium in Italy (10 mcg.), lately the EU aligned with the US, where there is no such requirement, so right now vanadium is no longer a trace element endowed with an RDA. We report it anyway, because vanadium has known properties, and because it could be reinserted in the official daily requirements list.

| | | AFA algae | | |
|---|---|---|---|---|
| Mineral | Other sources | 2 grams | Total | % RDA |
| Vanadium | 0 mg. | 6 mcg. | 6 mcg. | 60% |

Zinc

Zinc is an important trace mineral, insofar as it regulates normal biochemical functions in the body, such as supporting a healthy immune system, and acting as an enzyme activator in the many chemical reactions of our cells, our brain and the normal human development. Zinc is also important for the male reproductive system, and thus also to reduce the risk of prostate cancer. AFA algae contains a little quantity of zinc, at least in the relative terms of the 2-3 grams used as a supplement, approximately 80 mcg. for 2 grams. Thus, to produce a nutritional supplement as complete as possible based on AFA algae, we need to find a further source of zinc. Good candidates are extracts from oysters and other seashells (though quite expensive) , wheatgerm, nuts and seeds, and even an extract from micro algae such as AFA, or from Guava (Psidum Gujava) leaves, which can provide the RDA of 11 mg./day in just 250-300 mg.

| | | AFA algae | | |
|---|---|---|---|---|
| Mineral | Guava leaves ext 250 mg. | 2 grams | Total | % RDA |
| Zinc | 10 mg. | 80 mcg. | 10.08 mg. | 100% |

The Final Product

To conclude, the product that we have described uses AFA algae (or eventually Spirulina or Chlorella algae, though less nutrient-dense substitutes) as a base to be supplemented by other organic, natural extracts, each one rich in a certain vitamin or mineral in their organic and thus absorbable form. This product provides the full daily intake requirements, most of which at or above 100%, for all the vitamins and the most important micro and trace minerals, again in a form that is natural, organic, derived from food sources, and thus fully recognized by our body as food and consequently properly absorbed and utilized.

A possible version of this product, which is given here as an example and that does not exhaust the list of possible other versions (including different sources and concentrations), is the following, based on a sachet containing the following quantities of actives:

| | |
|---|---|
| AFA Klamath algae | 2,000 mg. |
| Extract of AFA algae and fruits | 180 mg. |
| (mango, lemon) - B vitamins | |
| Sunflower ext. - 15% Vit. E | 80 mg. |
| Moringa Oleifera extr. (0.1% Chromium) | 38 mg. |
| Curry leaves extract - Iron | 200 mg. |
| Dunaliella extr. - Betacarotene/vit. A | 100 mg. |
| Brassica Nigra seeds extr. - Selenium | 12 mg. |
| Guava leaves ext. - Zinc | 250 mg. |
| Acerola extr. - Vit. C | 120 mg. |
| Kelp extract - Iodine | 30 mg. |
| Mushrooms extr. - Vit D | 50 mg. |
| | 3,060 mg. |

### BIBLIOGRAPHY

Baroni L., Scoglio S. et al., Effect of a Klamath algae product (“AFA-B12”) on blood levels of vitamin B12 and homocysteine in vegan subjects: a pilot study, Int. J. Vitam. Nutr. Res., 79 (2), 2009, 117-123.

Heart Protection Study Collaborative Group (Oxford University), MRC/BHF Heart Protection Study of antioxidant vitamin supplementation in 20 536 high-risk individuals: a randomised placebo-controlled trial, in Lancet 2002; 360: 7-22.

Mayer D R et al., Essential trace elements in humans. Serum arsenic concentrations in hemodialysis patients in comparison to healthy controls, in Biol Trace Elem Res. 1993 April; 37(1):27-38.

Miller E. R. III et al, Meta-Analysis: High-Dosage Vitamin E Supplementation May Increase All-Cause Mortality, in Annals of Internal Medicine, 2005, 142: 37-46.

Omenn G S, Risk factors for lung cancer and for intervention effects in CARET the Beta-Carotene and Retinol Efficacy Trial, in J Natl Cancer Inst 1996 Nov. 6; 88(21):1550-9.

Pennington J. A., Young B. E., Total diet study nutritional elements, 1982-1989., J Am Diet Assoc. 1991 February; 91(2):179-83.

Syrad H. et al., Energy and nutrient intakes of young children in the UK: findings from the Gemini twin cohort. Br J Nutr. 2016 May 28; 115(10):1843-50.

Uthus E O., Effects of arsenic deprivation in hamsters, in Magnes Trace Elem. 1990; 9(4): 227-32;

1. A composition which always includes a nutritionally dense micro algae, such as but not limited to Aphanizomenon flos aquae, further supplemented by specific food and plant extracts rich in the natural, food derived vitamins and/or minerals that are lacking, or are insufficiently present in the same micro algae, in order to provide a multi-nutrients supplement as complete and as food based as possible.

2. A nutritional supplement according to claim 1 in which the micro algae is the Aphanizomenon flos aquae cyanobacterial micro algae from Klamath Lake, Oregon.

3. A nutritional supplement according to claim 1 whereby the micro algae selected is any other micro algae, cyanobacterial or not, which is fit for human consumption and nutritionally dense.

4. A nutritional supplement according to claim 1 , whereby the further source of natural, food derived Vitamin A (betacarotene) is an extract of Dunaliella Salina, Annatto seeds, sweet potato, carrots, kale, or any other food-source of Vitamin A/Betacarotene.

5. A nutritional supplement according to claim 1, whereby the further source of natural, food derived Vitamins B is an extract from Klamath AFA microalgae, leafy green vegetables, beans (green pea, lentils), fruit extracts (such as Guava, Mango, Lemon), Holy Basil or any other food rich in B vitamins.

6. A nutritional supplement according to claim 1, whereby the further source of natural, food derived Vitamin C is selected among extracts from fruits such as Acerola, Rosa Canina, Amla, and any other food rich in natural Vitamin C.

7. A nutritional supplement according to claim 1, whereby the source of natural, food derived Vitamin D is an extract of mushrooms, or any other food source of Vitamin D.

8. A nutritional supplement according to claim 1, whereby the further source of natural, food derived Vitamin E is an extract of soybeans, sunflower seeds, Annatto fruit (Bixa Oreliana), or any other dried oil and food representing a good source of vitamin E.

9. A nutritional supplement according to claim 1, whereby the further source of natural, food derived Chromium is an extract of grape juice, chromium-rich yeast, broccoli and/or Moringa Oleifera, sweet potato, apples, or any other natural food source of chromium.

10. A nutritional supplement according to claim 1, whereby the further source of natural, food derived Iron is an extract of beans, clams, blackstrap molasses, stinging nettle, pumpkin seeds, curry (Muraya koeinigii) leaves, or any other food rich in iron.

11. A nutritional supplement according to claim 1, whereby the further source of natural, food derived Selenium is an extract of mustard seeds, Brazilian nuts, Brassica Nigra, or any other foods rich in Selenium.

12. A nutritional supplement according to claim 1, whereby the further source of natural, food derived Zinc is an extract of oysters and seashells, or wheatgerm, or nuts and seeds, or Guava leaves.

13. A nutritional supplement according to claim 1, which is provided in different pharmaceutical forms, such as sachets, tablets, capsules or liquids.

14. A nutritional supplement according to claim 1 for human or animal consumption.

15. A nutritional supplement according to claim 1, whereby the further source of natural, food derived Iodine is an extract of the macro algae Kelp, or any other algae or food rich in iodine.

16. A nutritional supplement according to claim 1 whereby the micro algae is Spirulina.

17. A nutritional supplement according to claim 1 whereby the micro algae is Chlorella.

18. A nutritional supplement according to claim 1, whereby the micro algae used is a blend of the different microalgae.

19. A nutritional supplement according to claim 1, used to prevent or treat the nutritional deficiencies dependent on the lack of one or more of the nutrients provided by the supplement.

20. A nutritional supplement according to claim 1, where the supplement is used to prevent or treat the human or animal pathologies associated with said nutritional deficiencies.
